SEQUENCE LISTING (3 sequences)

SEQ ID NO: 1 - DNA, 22 bp, Raffaelea lauricola
cgagtgaagcggcaacagctca

SEQ ID NO: 2 - DNA, 21 bp, Raffaelea lauricola
cgccgccagaagcgtcctctc

SEQ ID NO: 3 - DNA, 21 bp, Raffaelea lauricola
ccgcgggcccgagttgtactt

(19) United States
(12) Patent Application Publication
(10) Pub. No.: US 2016/0273055 A1
(43) Pub. Date: Sep. 22, 2016

(54) NANOPARTICLE BASED ASSAY TO DETECT FUNGAL INFECTION

(71) Applicants: DeEtta MILLS, Homestead, FL (US); Bruce MCCORD, Miami, FL (US); Annia MESA, Miami, FL (US); Christina BURNS, Miami, FL (US)

(72) Inventors: DeEtta MILLS, Homestead, FL (US); Bruce MCCORD, Miami, FL (US); Annia MESA, Miami, FL (US); Christina BURNS, Miami, FL (US)

(73) Assignee: The Floria International University Board of Trustees, Miami, FL (US)

(21) Appl. No.: 14/660,303

(22) Filed: Mar. 17, 2015

(51) Int. Cl. C12Q 1/68

(52) U.S. Cl. CPC C12Q 1/6895; C12Q 2600/158

(57) ABSTRACT

The invention pertains to a nanoparticle based assay for detecting the presence of a target nucleic acid specific to a fungus. The assay can comprise the steps of obtaining nucleic acids from the sample, contacting the nucleic acids from the sample with a single stranded nucleic acid (ssNA) probe complementary to a target nucleic acid specific for the fungus, and adding nanoparticles to the mixture. The presence of the target nucleic acid and hence the presence of the fungus in the sample is indicated by a particular color associated with aggregated nanoparticles in the solution; whereas, the absence of the target nucleic acid and hence the absence of the fungus in the sample is indicated by a different color associated with dispersed nanoparticles in the solution. Kits comprising nanoparticles and/or ssNA probe are also provided.

The Sequence Listing for this application is labeled “SeqList-17Mar15-ST25.txt”, which was created on Mar. 17, 2015, and is 1 KB. The entire content is incorporated herein by reference in its entirety.

### BACKGROUND OF THE INVENTION

Laurel wilt disease is a vascular disease caused by a fungal phytopathogen, such as Raffaelea lauricola. This disease is devastating both to the wild laurels and the commercially important avocado trees. For example, this disease is seen in wild laurels on the US east coast and commercial avocado trees in South Florida. Recently the laurel trees in Mississippi have shown signs of wilt disease. The disease may continue to move westward and threaten other laurel species and the important commercial avocado industries in California and Mexico.

Currently, it takes up to two weeks from the time a sample is submitted to a service laboratory to confirm the presence of the fungus causing a disease because the sample must first be cultured in the laboratory. Nucleic acids are then extracted from the cultures for identification of the fungus.

By the time the results confirm the presence of the pathogen, the tree may be dead and the fungus spread to surrounding trees. Such spread of the disease is devastating to the industry.

### BRIEF SUMMARY OF THE INVENTION

The invention provides efficient and quick methods for the identification of a fungus in a sample such as a plant tissue.

In one embodiment, the invention provides a nanoparticle based assay for detecting the presence of a target nucleic acid specific to a fungus in a sample. The assay can comprise the steps of obtaining nucleic acids from the sample, contacting the nucleic acids obtained from the sample with a single stranded nucleic acid (ssNA) probe complementary to a target nucleic acid specific for the fungus, and adding nanoparticles to the mixture. The presence of the target nucleic acid in the nucleic acids obtained from the sample, and hence the presence of the fungus in the sample, is indicated by a particular color associated with aggregated nanoparticles in the solution; whereas, the absence of the target nucleic acid in the nucleic acids obtained from the sample, and hence the absence of the fungus in the sample, is indicated by a different color associated with dispersed nanoparticles in the solution.

The nucleic acids obtained in a sample can be amplified by, for example, polymerase chain reaction using primers specific for the target nucleic acid. The nucleic acid obtained from a sample can be genomic DNA and/or RNA isolated from the sample.

The invention also provides kits to conduct the nanoparticle based assays provided herein. The kits can comprise nanoparticles and ssNA probes. The kits can also comprise primers specific for the fungus and/or reagents for isolating nucleic acids from the sample. The kits can further comprise reagents for preparing the reaction mixtures, reagents for carrying out polymerase chain reaction, sodium chloride or a solution of sodium chloride, and hybridization buffer or constituents of a hybridization buffer.

### BRIEF DESCRIPTION OF THE DRAWINGS

FIG. 1 illustrates a schematic representation of the principle of the nanoparticle based assay.

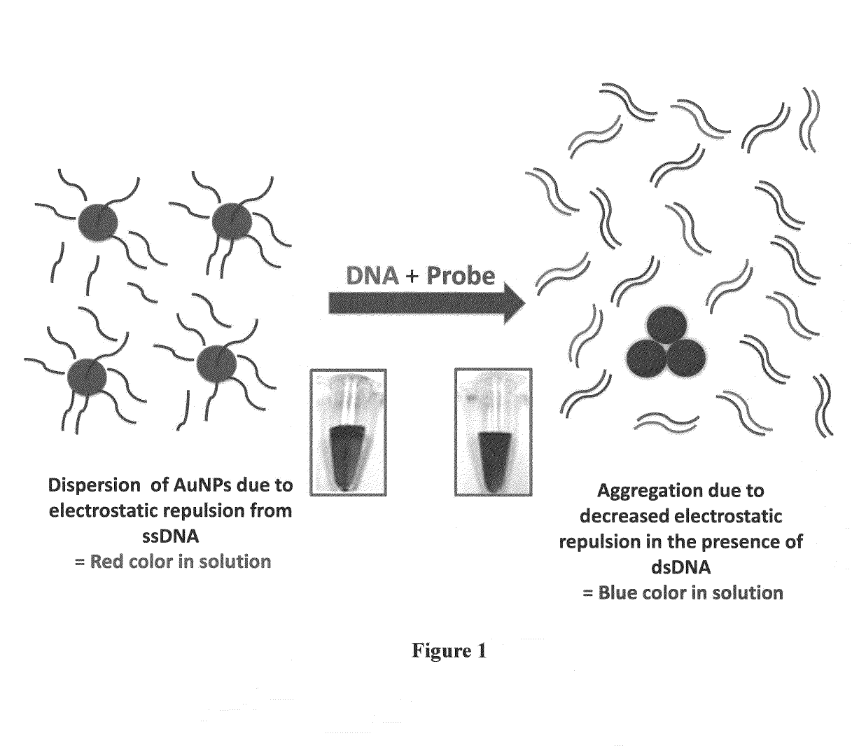

FIG. 2 illustrates a protocol for conducting an embodiment of the nanoparticle based assay.

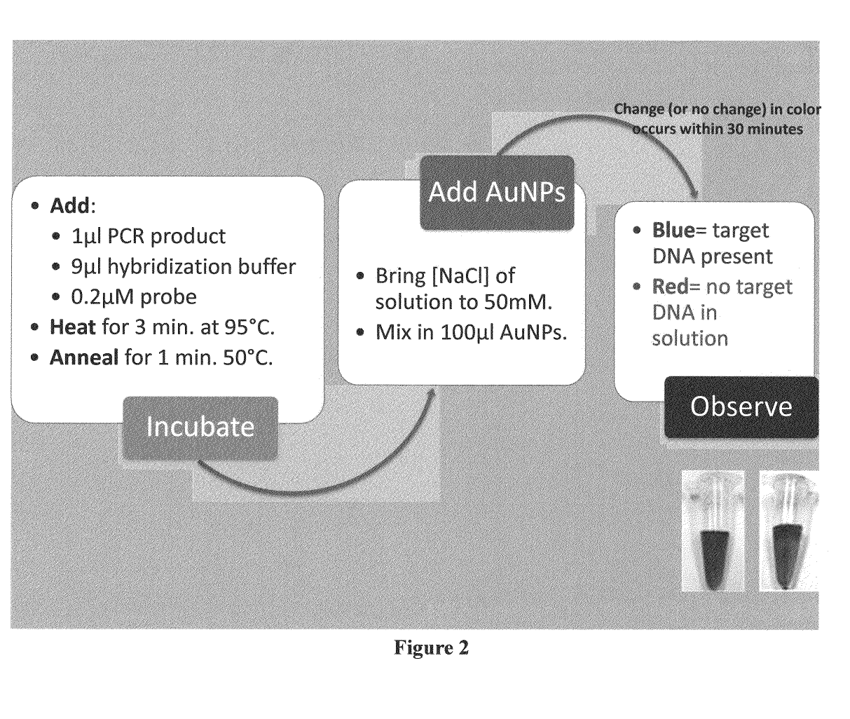

### BRIEF DESCRIPTION OF THE SEQUENCES

SEQ ID NO: 1: Sequence of the forward primer for amplification of 28S RNA from R. lauricola.

SEQ ID NO: 2: Sequence of the reverse primer for amplification of 28S RNA from R. lauricola.

SEQ ID NO: 3: Probe sequence for identification of 28S RNA amplification product from R. lauricola.

### DETAILED DESCRIPTION OF THE INVENTION

The subject invention provides efficient methods for detecting fungal pathogens, such as R. lauricola, based on a nanoparticle based biosensor assay. In specific embodiments, nanoparticle biosensors, for example, gold nanoparticles (AuNPs) or silver nanoparticles (AgNP) biosensors can be used in the methods of the subject invention.

Certain nanoparticles produce a different colored solution when dispersed in the solution versus when they aggregate. The assay of the current invention is based on two principles: (a) in the presence of ssNA probe, for example, in a high salt solution, the dispersion of nanoparticles, for example, AuNP or AgNP is stabilized; whereas, (b) if the ssNA probe is hybridized to its complement, for example, the genomic DNA or RNA from a sample, the dispersion stability of the ssNA probe is removed and the nanoparticles (for example, AuNPs or AgNPs) aggregate.

Accordingly, the invention provides a method of detecting a fungus, for example, a fungal pathogen, in a sample, for example, a plant tissue. The method comprises contacting nucleic acids obtained from the sample with an ssNA probe complementary to a target nucleic acid specific for the fungus to be detected.

The step of contacting nucleic acids obtained from the sample with an ssNA probe complementary to a target nucleic acid specific for the fungus to be detected is performed under conditions that allow hybridization of the ssNA probe with the target nucleic acid. Therefore, if the nucleic acid obtained from the sample contains the target nucleic acids the ssNA probe binds to the target nucleic acids.

The method further comprises adding nanoparticles, for example, AuNPs or AgNPs, to the mixture of ssNA and the nucleic acids obtained from the sample. Upon sufficient incubation time, the solution of nanoparticles, ssNA, and nucleic acids obtained from the sample exhibits a specific color depending on the complementarity between the ssNA and the nucleic acids obtained from the sample. Particularly, if the nucleic acids from the sample and the ssNA are complementary to each other, the ssNA probe is removed from solution (i.e., it hybridizes to its target) and a solution of a particular color is produced indicative of aggregation of nanoparticles. If hybridization does not take place between the ssNA and the nucleic acids obtained from the sample (i.e., the target nucleic was not present, thus the probe did not hybridize), a different colored solution is produced as the ssNA probes stabilize the dispersed nanoparticles.

In one embodiment, the sample is a plant tissue and the fungus is R. lauricola. Accordingly, the invention provides a method of detecting R. lauricola infection in a plant. Any plant tissue, for example, wood, leaves, roots, inflorescence can be used according to the methods of the current invention.

In one embodiment, the nucleic acid obtained from the sample is amplified by, for example, a polymerase chain reaction. For example, a fragment of the nucleic acids having a sequence unique to the fungus being detected can be amplified using specific primers by PCR. In an embodiment, the fragment of nucleic acids unique to the fungus being detected corresponds to the fungal 28S ribosomal RNA. Accordingly, the nucleic acids obtained from the sample are PCR amplified using primers specific for the 28S ribosomal RNA from the fungus being detected.

In another embodiment, the nucleic acids isolated from the sample are the genomic DNA and/or RNA isolated from the sample. In one embodiment, the DNA and/or RNA is isolated from the sample by using Filter Paper Technology paper (FTA paper). In this embodiment, the filter paper is chemically treated to lyse fungal cells that are placed on the FTA paper. For example, a fungal culture can be smeared onto the paper, which lyses the cells and also preserves the sample at room temperature. In one embodiment, an FTA Elute paper is used. FTA Elute paper performs the cell lysis in the same manner as the FTA paper but allows for the genomic DNA/RNA to be eluted from the paper. The genomic DNA/RNA can then be tested without having to amplify the target nucleic acid using PCR.

The ssNA probe can be a single stranded DNA (ssDNA) probe or single stranded RNA (ssRNA) probe. In one embodiment, the ssNA is single stranded XNA (ssXNA, nucleic acid analogs or artificial nucleic acids). Examples of XNA include, but are not limited to, peptide nucleic acid (PNA), Morpholino and locked nucleic acid (LNA), glycol nucleic acid (GNA), and threose nucleic acid (TNA).

In one embodiment, the ssNA probe hybridizes to its target within the nucleic acids obtained from the sample, for example, genomic DNA or RNA, or PCR amplified target DNA obtained from the sample. The step of contacting is performed under conditions that allow hybridization of the ssNA probe with the target nucleic acid. Particularly, hybridization between the ssNA probe and the target nucleic acids can be controlled by modifying the conditions that allow binding between nucleic acids having high complementarity (high stringency conditions), intermediate complementarity (intermediate stringency), or low complementarity (low stringency). Various degrees of stringency of hybridization can be employed. The more severe the conditions, the greater the complementarity required for the binding between the ssNA and the target nucleic acids. Severity of conditions can be controlled by temperature, probe concentration, probe length, ionic strength, time, etc. For example, higher temperatures typically allow binding between nucleic acids having high complementarity and vice versa. Also, high probe concentration allows binding between nucleic acids having low complementarity and vice versa. Further, high salt concentration allows binding between nucleic acids having low complementarity and vice versa. A person of ordinary skill in the art can determine appropriate conditions for a particular purpose.

In one embodiment, unbound nucleic acids can be digested to remove any ssNA probes or single stranded fungal DNA or RNA prior to mixing the nanoparticles.

In certain embodiments, the ssNA probe is specific for a fungal pathogen to be detected. In one embodiment, the fungal pathogen is R. lauricola. A person of ordinary skill in the art can design a test according to the current invention to detect any fungal pathogen based on a nucleic acid sequence unique to the fungal pathogen of interest and such embodiments are within the purview of the current invention.

In certain embodiments, the nanoparticles that can be used in the current invention are metal nanoparticles. Non-limiting examples of metal nanoparticles include gold, silver, titanium, platinum, iron, molybdenum, manganese, nickel, cobalt, palladium, tin, zinc, lead, copper, aluminum, and alloys thereof.

The metal nanoparticles that are suitable for use in the current invention exhibit a color change depending on the size and aggregation of the nanoparticles in the solution. For example, a solution containing aggregated nanoparticles has a different color compared to a solution containing dispersed nanoparticles. For example, when AuNPs are aggregated a blue solution is produced; whereas, when AuNPs are dispersed, a red solution is produced. Similarly, when AgNPs are aggregated a grey/black solution is produced; whereas, when AgNPs are dispersed, a yellow solution is produced.

A person of ordinary skill in the art can determine color change based on aggregation of a particular nanoparticle to determine which nanoparticles of various metals having various sizes are suitable for use according to the methods described herein and such embodiments are within the purview of the invention.

The size of the nanoparticles used in the methods described herein can be adjusted based on a particular nanoparticle used. Typically, the size of the nanoparticles is about 10 nm to about 100 nm, about 20 nm to about 80 nm, about 30 nm to about 70 nm, about 40 nm to about 60 nm, about 10 nm to about 20 nm, about 20 nm to about 30 nm, about 30 nm to about 40 nm, about 50 nm to about 60 nm, about 60 nm to about 70 nm, about 70 nm to about 80 nm, about 80 nm to about 90 nm, or about 90 nm to about 100 nm.

Further embodiments of the invention provide kits for conducting the methods of the current invention. The kit can comprise nanoparticles and ssNA probes specific for a fungus to be detected. The kit can also contain primers specific for a fungus to be detected and/or reagents for isolating genomic DNA and/or RNA from a sample. The kit can further comprise reagents for carrying preparing the reaction mixtures, for example, sodium chloride solution or hybridization buffer. Alternately, the kit can comprise solid sodium chloride or constituents of the hybridization buffer which can be used to produce sodium chloride solution or hybridization buffer. The kit can also contain reagents for carrying out PCR.

Various apparatuses used to conduct the assay, for example, reaction tubes, can also be provided with the kit. In one embodiment, the kit comprises instruction materials for conducting the assay.

The term “about” is used in this patent application to describe some quantitative aspects of the invention, for example, size of nanoparticles. It should be understood that absolute accuracy is not required with respect to those aspects for the invention to operate. When the term “about” is used to describe a quantitative aspect of the invention the relevant aspect may be varied by ±10%.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### AuNP Synthesis

The Turkevich reaction, or citrate reduction method, involves reduction of a hydrogen tetrachloroaurate (HAuCl₄) solution via addition of sodium citrate. After HAuCl₄ and water are brought to a low boil, sodium citrate is added and the reduction mixture is refluxed for 30 minutes. Once it is cooled to room temperature, the solution is filtered using a 0.45 μm syringe filter. The resulting solution contains a dispersion of AuNPs with a dark red color.

Cooling and storage procedures can be adjusted to accommodate particle size considerations for protocol optimization.

The reaction is relatively simple to execute and adjusting the amount of sodium citrate used allows for different particle sizes to be synthesized.

### Example 2

### DNA Extraction from Wood Samples or Fungal Cultures

DNA extraction for wood samples or fungal cultures was done by using the FastDNA™ SPIN KIT for SOILS (MP Biomedicals) following the manufacturer's protocol. The DNA was quantified using the Qubit 2.0 Fluorometer.

Genera-specific primer sets and complementary probes were identified for R. lauricola and close relatives as described in Jeyaprakash et al. (2014).

Accordingly, the primers and probes used are:

Forward primer:
LW28S-F1
(SEQ ID NO: 1)
(5′-CGAGTGAAGCGGCAACAGCTCA-3′)
Reverse primer:
LW28S-R1
(SEQ ID NO: 2)
(5′CGCCGCCAGAAGCGTCCTCTC-3′)
ssDNA probe:
LW28Sp
(SEQ ID NO: 3)
([6FAM]-CCGCGGGCCCGAGTTGTACTT-[BHQ])

All primers and probes are purchased from Integrated DNA Technologies, Inc.

### Example 3

### The PCR Based Biosensor Assay

The PCR product was added to the hybridization buffer containing ssDNA probe LW28Sp, phosphate buffer, and NaCl. The mixture was then be subjected to denaturation for 3 min at 95° C. followed by 1 min for probe annealing at 50° C. Because high temperatures can lead to AuNP aggregation, the unmodified AuNPs were added to the reaction mix only after the annealing step of the PCR product to the probe is completed.

If the probe binds to its complementary sequence on the PCR product, then the AuNPs (left destabilized in the absence of ssDNA probe adsorption) aggregate and change to a blue color indicating a positive result for the presence of R. lauricola. If the PCR product did not amplify R. lauricola DNA the probe cannot hybridize to its complementary sequence and the AuNP stayed dispersed and a red color indicates the absence of R. lauricola DNA.

### Example 4

### Genomic DNA Based Biosensor Assay

This embodiment provides a method for the collection and extraction of fungus, for example, R. lauricola, genomic DNA for analysis with AuNPs. In this method, genomic DNA was extracted from a pure culture sample that is swabbed onto the cellulytic filter paper of FTA® Elute Cards (Whatman®). Cell lysis releases genomic DNA which is trapped on the paper. The genomic DNA was later be eluted from the paper and was used to test with the AuNP test. The Whatman® protocol for eluting the DNA from the filter paper was followed.

This method eliminates the DNA extraction step and even the amplification step required of the current pathogen detection protocol. The eluted genomic DNA is targeted by ssDNA probes. The AuNP test results from this approach can provide comparable results with that of the PCR product-probe approach. This method allows for a rapid, sensitive and species-specific AuNP biosensor for R. lauricola.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Khalil et al., (2014), A sensitive colorimetric assay for identification of Acinetobacter baumannii using unmodified gold nanoparticles, Journal of Applied Microbiology, Vol. 117, Issue 2, pp. 465-471.
2. Xia et al., (2010), Colorimetric detection of DNA, small molecules, proteins, and ions using unmodified gold nanoparticles and conjugated polyelectrolytes, PNAS, Vol. 107, No. 24, pp. 10837-10841.
3. Jeyaprakash et al., (2014), Molecular Detection of the Laurel Wilt Fungus, Raffaelea lauricola, Plant Disease, Vol. 98, No. 4, pp. 559-564.

1. A method of detecting the presence of a fungus in a sample, the method comprising the steps of:
a. contacting nucleic acids obtained from the sample with a single stranded nucleic acid (ssNA) probe complementary to a target nucleic acid specific for the fungus, said contacting performed under conditions that allow hybridization of the ssNA probe with the target nucleic acid,
b. adding nanoparticles to the mixture of ssNA and the nucleic acids obtained from the sample,
c. incubating the resulting mixture comprising nanoparticles, ssNA, and the nucleic acids obtained from the sample, and
d. identifying the presence of the fungus in the sample if the resulting mixture exhibits a color indicative of aggregation of the nanoparticles, or
identifying the absence of the fungus in the sample if the resulting mixture exhibits a color indicative of dispersion of the nanoparticles.

2. The method of claim 1, wherein the nucleic acids obtained from the sample comprise a polymerase chain reaction (PCR) amplified fragments produced from the nucleic acids isolated from the sample.

3. The method of claim 2, wherein the nucleic acids obtained from the sample are PCR amplified fragments produced from the nucleic acids isolated from the sample by using primers specific for the 28S ribosomal RNA from the fungus.

4. The method of claim 1, wherein the fungus is R. lauricola.

5. The method of claim 3, wherein the fungus is R. lauricola and the primers specific for the 28S ribosomal RNA comprise the sequences of SEQ ID NO: 1 and SEQ ID NO: 2.

6. The method of claim 5, wherein the ssNA probe has the sequence of SEQ ID NO: 3.

7. The method of claim 1, wherein the ssNA probe is a single stranded DNA (ssDNA) probe, single stranded RNA (ssRNA) probe, or a single stranded nucleic acid analogs or artificial nucleic acid (ssXNA) probe.

8. The method of claim 7, wherein the XNA probe is single stranded peptide nucleic acid probe (ssPNA), single stranded morpholino and locked nucleic acid probe (ssLNA), single stranded glycol nucleic acid probe (ssGNA) or a single stranded threose nucleic acid probe (ssTNA).

9. The method of claim 1, wherein nucleic acids obtained from the sample are the genomic DNA and/or RNA isolated from the sample.

10. The method of claim 9, wherein the genomic DNA and/or RNA is isolated by a Filter Paper Technology (FTA paper) Elute based method.

11. The method of claim 9, the method further comprising the step of digesting the mixture of ssNA and the genomic DNA and/or RNA isolated from sample to remove free ssNA probes or single stranded DNA or RNA prior to adding the nanoparticles to the mixture of ssNA and the genomic DNA and/or RNA.

12. The method of claim 1, wherein the nanoparticles are metal nanoparticles.

13. The method of claim 12, wherein the metal is gold, silver, titanium, platinum, iron, molybdenum, manganese, nickel, cobalt, palladium, tin, zinc, lead, copper, aluminum, or an alloy thereof.

14. The method of claim 1, wherein the nanoparticles are gold nanoparticles (AuNPs) and blue color is indicative of aggregation of the AuNPs; whereas, red color is indicative of dispersion of the AuNPs.

15. The method of claim 1, wherein the nanoparticles are silver nanoparticles (AgNPs) and grey/black color is indicative of aggregation of the AgNPs; whereas, yellow color is indicative of dispersion of the AgNPs.

16. The method of claim 1, wherein the sample is a plant tissue.

17. A kit comprising:
a. nanoparticles,
b. ssNA probe specific for a fungus,
c. optionally, nucleic acid primers specific for the fungus, and
d. optionally, reagents for isolating genomic DNA and/or RNA from a sample.

18. The kit of claim 17, wherein the nanoparticles are AuNPs or AgNPs, the ssNA probe comprises the sequence of SEQ ID NO: 3, and the nucleic acid primers comprise the sequences of SEQ ID NO: 1 and 2.

19. The kit of claim 18, the kit further comprising reagents for carrying out PCR.

20. The kit of claim 19, the kit further comprising a hybridization buffer or components of the hybridization buffer.
